(19) United States
(12) Patent Application Publication
(10) Pub. No.: US 2010/0324431 A1
(43) Pub. Date: Dec. 23, 2010

(54) Determining Disease State Using An Induced Load

(75) Inventors: Paul Stanley Addison, Edinburgh (GB); James Nicholas Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/487,470

(22) Filed: Jun. 18, 2009

(51) Int. Cl. A61B 5/02

(52) U.S. Cl. 600/504

(57) ABSTRACT

The present disclosure relates to determining a patient's disease state based at least in pail on obtaining or determining certain underlying characteristics, such as vasotone, venous compliance, or ability of the vascular system to drain venous blood, of the patient's vascular system. The characteristics may be obtained by analyzing changes to a patient signal, such as the overall signal change, the rate of change, the shape of the change, changes in signal energy, or changes in the baseline and/or the amplitude of the signal, and/or the time period(s) over which the signal changes, that are caused by inducing a load on the vascular system. In some embodiments, the signal changes may be analyzed by transforming the signal using, for example, a continuous wavelet transform. The patient's health status or disease state may be determined using the one or more vascular system characteristics that influenced the signal change.

### SUMMARY

The present disclosure relates to patient monitoring, and more particularly, relates to determining a patient's health status or susceptibility to a disease state.

It may be important to determine or obtain certain underlying characteristics of a patient's vascular system in a clinical setting. Information about one or more characteristics of the vascular system may be gleaned from analyzing a patient signal that changes due to inducing a load on the vascular system. For example, the patient signal may change if the patient elevates or lowers a limb to which a patient sensor is attached. Other examples of when the patient signal may change include a patient holding his or her breathe, a patient breathing against a known resistance and/or creating a known pressure in the lungs, a patient lying down then standing up, a patient undergoing exercise (e.g. running or cycling), a patient ceasing exercise, or any suitable combination of these events. In some embodiments, one or more vascular system characteristics may influence such signal changes as the overall change in the signal, the rate of change of the signal, the shape of the signal change including any individual pulse changes (e.g., when analyzing a plethysmograph signal or other physiological signal containing repetitive features), changes in signal energy, or changes in the baseline and/or the amplitude of the signal, and/or the time period(s) over which the signal changes. In some embodiments, signal changes may be analyzed by transforming the signal using, for example, a continuous wavelet transform. The patient's health status or disease state also may be determined as a result of obtaining one or more vascular system characteristics that may influence the signal change. For example, vascular characteristics such as the patient's vasotone, venous compliance, or ability of the vascular system to drain venous blood, may be used to diagnose or predict the patient's current or future health or susceptibility to particular diseases.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and Illustrative schematics of a further wavelet decomposition of these newly derived signals in accordance with an embodiment;

FIGS. 3(e) and 3(f) are flow charts of illustrative steps involved in performing an inverse continuous wavelet transform in accordance with an embodiment;

FIG. 4 is a block diagram of an illustrative continuous wavelet processing system in accordance with an embodiment;

FIG. 5 shows a graphical illustration of a PPG signal obtained from a patient in accordance with an embodiment;

FIG. 6(a) shows PPG signal changes induced by varying a load in a patient's vascular system in accordance with an embodiment;

FIG. 6(b) shows an enlarged portion of the PPG signal in FIG. 6(a) in accordance with an embodiment;

FIG. 6(c) shows an illustrative scalogram derived firm the PPG signal of FIG. 6(a) in accordance with an embodiment;

FIGS. 7(a)-(b) show graphical illustrations of PPG signal changes induced by a load in a patient's vascular system in accordance with an embodiment;

FIG. 8 is a flow chart of an illustrative process for determining a disease state using an induced load in accordance with an embodiment; and

FIG. 9 is a flow chart of an illustrative process for communicating a disease state in a vascular system using a PPG signal in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=I₀(λ)exp(−(sβ₀(λ)+(1−s)βᵣ(λ))/(t)) (1)

where:
λ=wavelength;
t-time;
I=intensity of light detected;
I₀=intensity of light transmitted;
s=oxygen saturation;
β₀, βᵣ=empirically derived absorption coefficients; and
1(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log I₀−(sβ₀+(1−s)βᵣ)I (2)
2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) \text{/} d t}{d log I ( {λ}_{IR} ) \text{/} d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A-log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂, λ_{IR})−I(t₁, λ_{IR})]I(t₁, λ_{R})

y(t)=[I(t₂, λ_{R})−I(t₂, λ_{R})]I(t₁, λ_{IR}) (8)

y(t)=Rx(t)

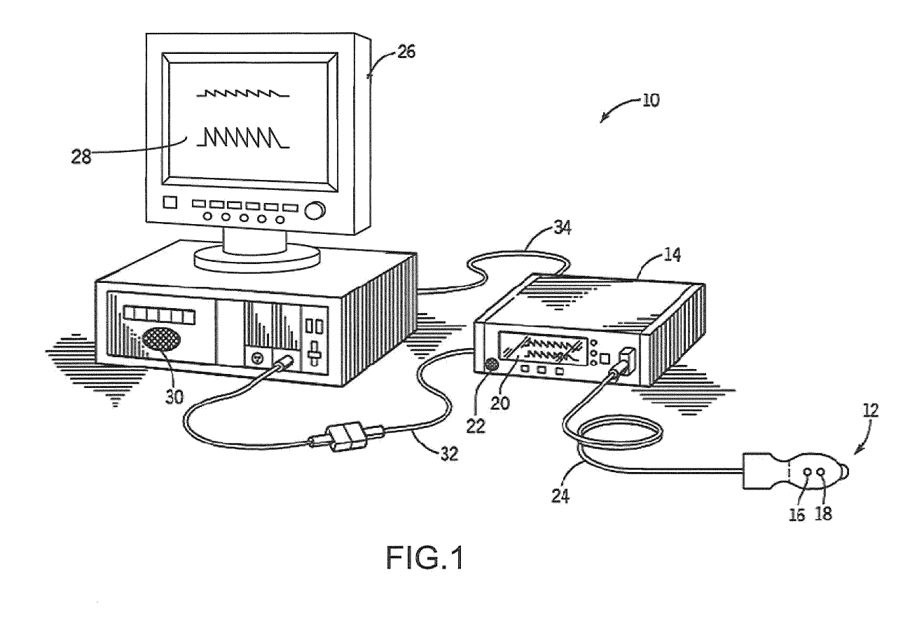

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

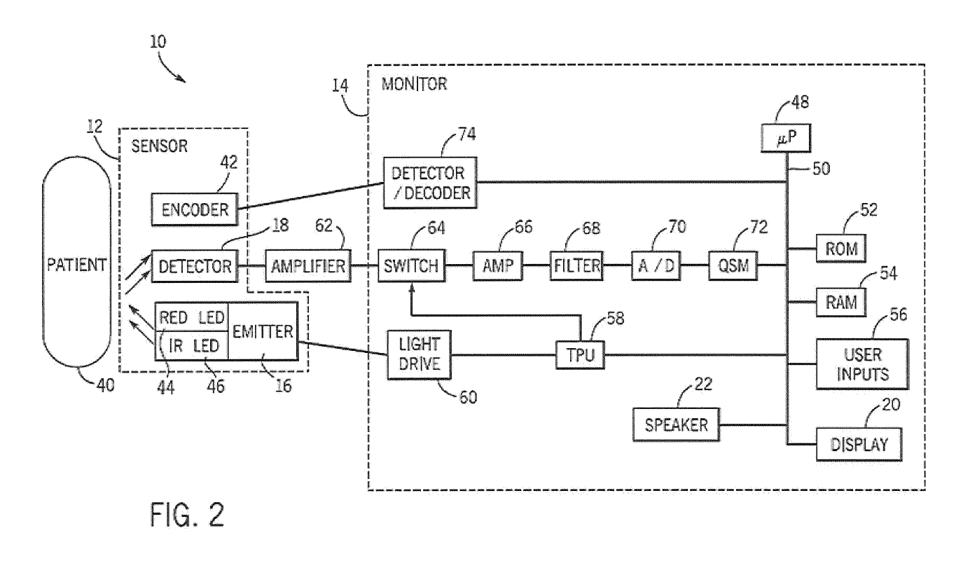

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal S on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence) operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear rescaling, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet is defined as:

ψ(t)=π^{-I/4}(e^{i2πf}^{ₜ}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

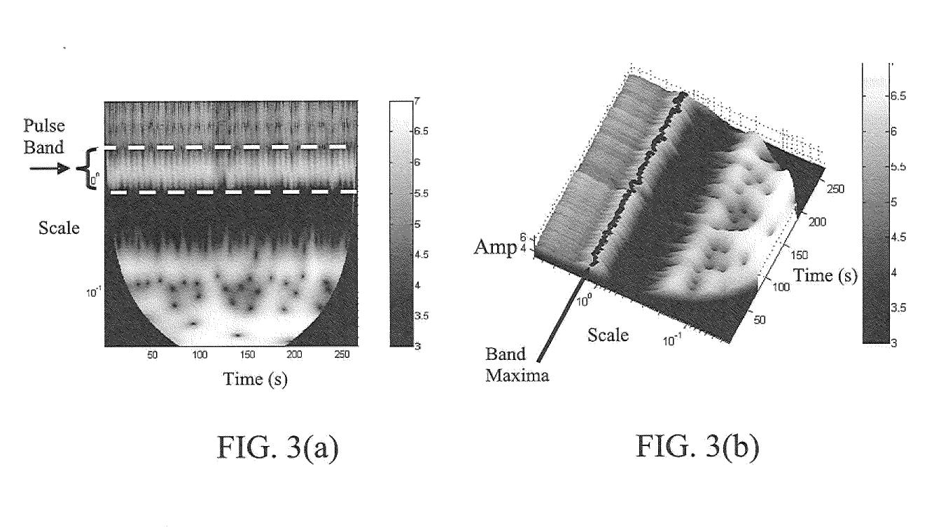

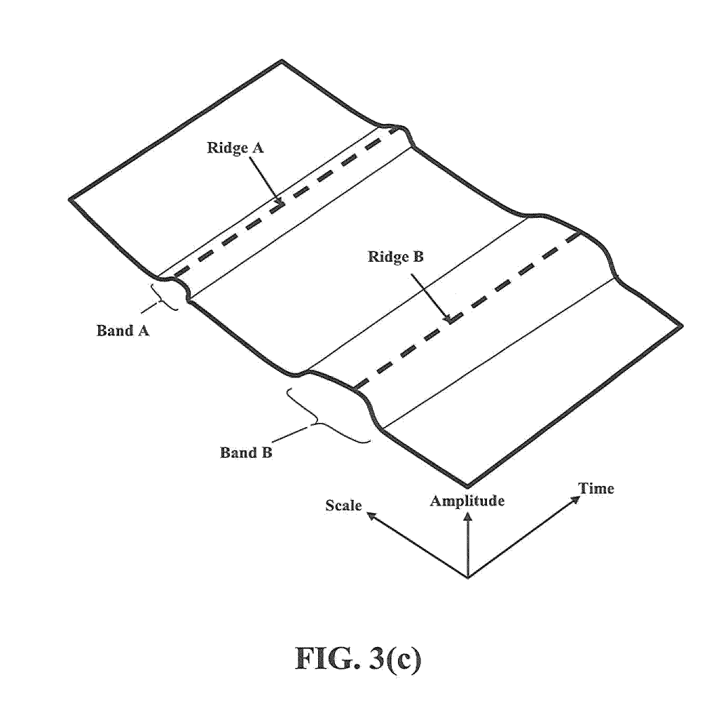

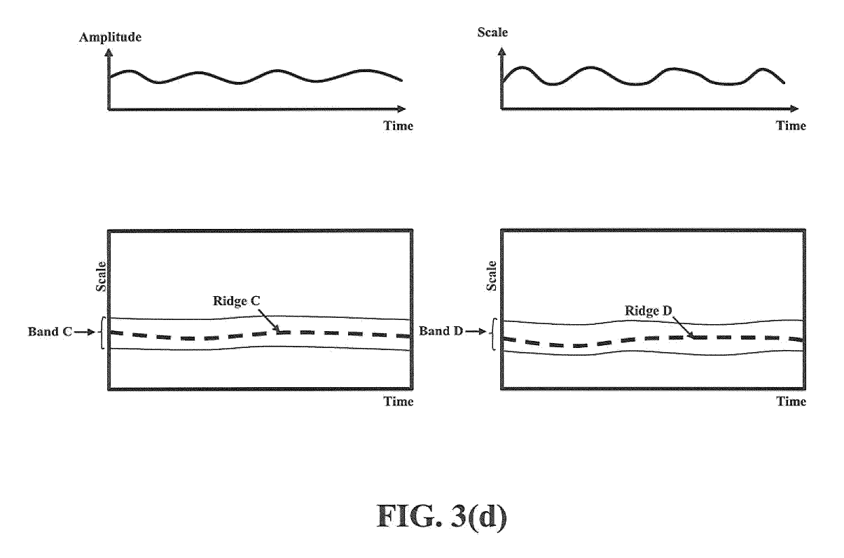

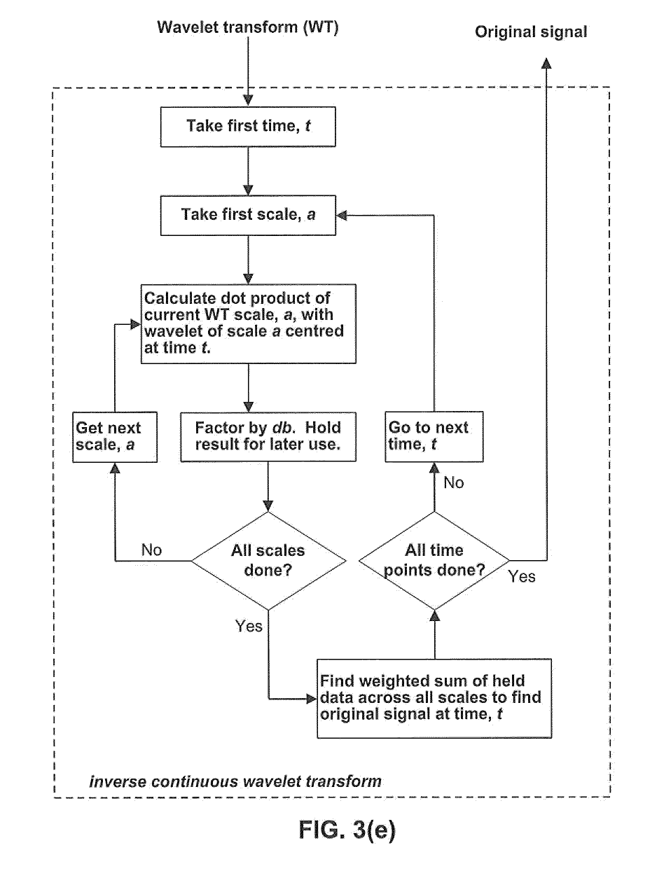

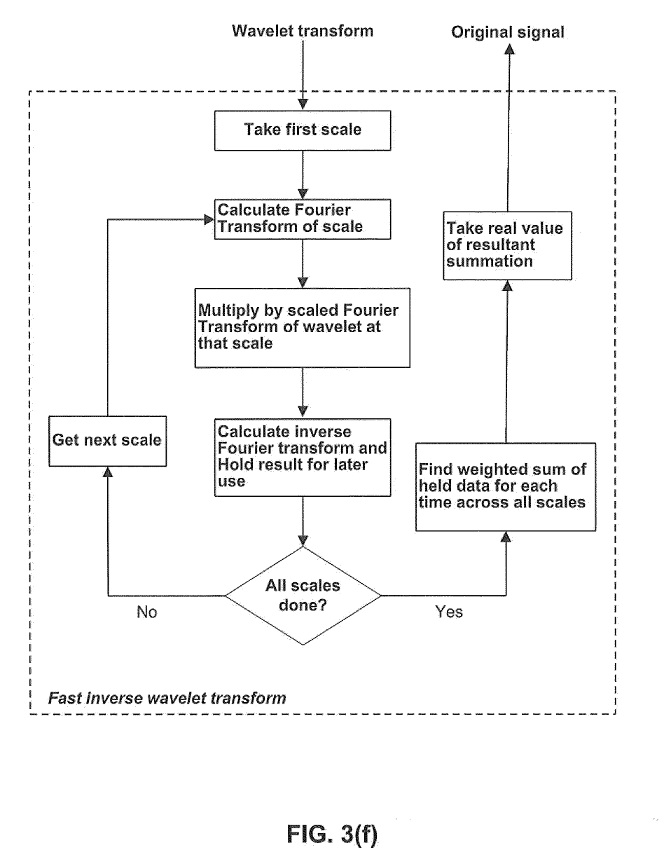

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

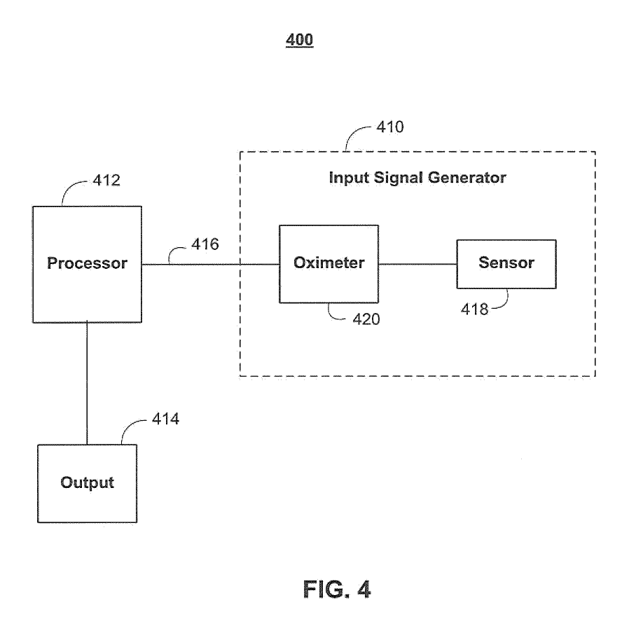

FIG. 4 is an illustrative continuous wavelet processing system 400 in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14. It will be further understood that system 400 and/or system 10 may be used with any other suitable biosignal (e.g., an arterial line blood pressure signal, patient signals sensed using two sensors 12 placed at any suitable location, such as a finger and a forehead or ear (e.g., for continuous non-invasive blood pressure (CNIBP) measurement), an EKG signal used to both determine one or more characteristics of the patient's vascular system and monitor the patient's health status during any induced load on the vascular system, any other suitable signal, or any combination thereof) sensed from patient 40 to obtain one or more characteristics of the patient's vascular system and to determine the patient's susceptibility to current or future diseases. For example, microprocessor 48 or processor 412 may obtain the patient's vascular characteristics and/or disease susceptibility using various processes and/or look-up tables based on the value of the received signal changes and/or data.

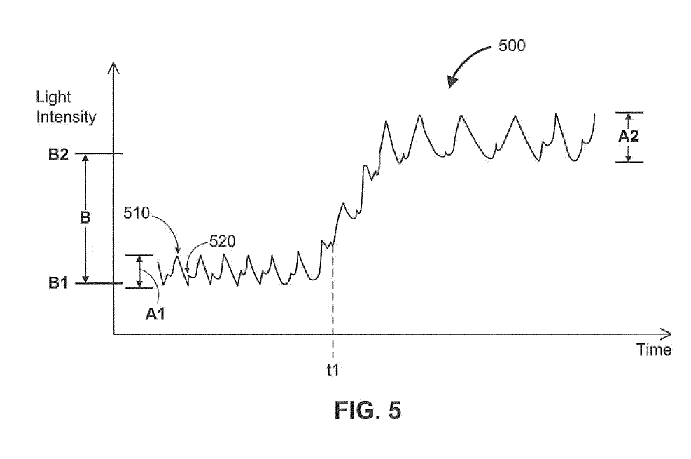

It may be important to determine or obtain certain characteristics of a patient's vascular system in a clinical setting and to determine how those characteristics may predict the patient's health status or disease state. For example, the static and dynamic characteristics underlying the patient's vascular system may be used in diagnosing or predicting the patient's susceptibility to particular vascular diseases. Information about the characteristics of the patient's vascular system may be gleaned from analyzing changes induced in a signal (e.g., a PPG signal) sensed by a sensor coupled to patient 40. The signal changes may be induced by introducing any suitable load to the vascular system, including, but not limited to, changing the elevation of the sensor relative to patient 40 (e.g., by lifting or lowering the hand and finger on which the sensor is coupled to patient 40 relative to the sensor's initial, or resting, location). Signal changes that may be induced by the load and may be influenced by the vascular system characteristics include the overall change in the signal, the rate of change of the signal, the shape of the signal pulse (e.g., the washing out, or diminishing, of the dicrotic notch in a PPG signal), changes in signal energy, changes in the baseline and/or the amplitude of the signal, and the time period(s) over which the signal change may occur. The vascular system characteristics that influence the signal changes may also indicate the patient's future health status or disease susceptibility. It will be understood that the present disclosure may be applied to inducing any suitable load in any suitable biosignal (e.g., electrocardiogram, heart rate signal, plethysmograph signal, or any other suitable signal). However, for brevity and clarity, certain embodiments are described below in terms of inducing loads via elevation changes of the sensor to influence PPG signal changes, determine one or more characteristics of the patient's vascular system, and determine the patient's disease state. Embodiments will now be discussed in connection with FIGS. 5-9.

A load may be induced in any suitable signal, such as a PPG signal, to obtain characteristics of a patient's vascular system. FIG. 5 shows a graphical illustration of PPG signal 500 obtained over time from a patient in accordance with an embodiment. PPG signal 500 may be sensed using any suitable means, such as sensor 418 or sensor 12, and may represent the light intensity detected by the sensor over time after the light passes through the patient's tissue (e.g., the patient's finger). PPG signal 500 may include a mean baseline value of B1 and an amplitude of A1 when the patient is stable or at rest (e.g., when the patient's finger is level with the patient's heart). Each pulse of PPG signal 500 may correspond to one cardiac cycle and may include a primary peak 510 and a dicrotic notch 520.

PPG signal 500 may change when a load is induced on the patient's vascular system. For example, at time t1, the patient may raise the hand to which sensor 12 may be coupled, which may change the baseline and the amplitude of PPG signal 500. The baseline of PPG signal 500 may increase by an amount B to a new baseline mean B2 as the venous blood drains from the raised limb, including the finger to which sensor 12 may be coupled. The amplitude of PPG signal 500 may be A2 after the limb is elevated, where A2 may be greater than initial amplitude A1, as the vessels in the elevated limb become more free to expand because the elevated limb contains less blood at lower pressure. If the patient were to lower the limb to which sensor 12 is coupled below the initial resting position, then the opposite effect may be observed; the baseline and amplitude of PPG signal 500 may decrease as venous pooling of the patient's blood occurs and less light may be transmitted through the patient's tissue. Changing the elevation of the limb may also change the patient's heart rate (e.g., due to strenuous exercise or the effect of inducing the load). Neither the baseline nor the amplitude changes may be immediately detectable in PPG signal 500 following the induction of the load (e.g., the limb movement), but may occur within a transition period following the induction. Although introducing a load to a patient's vascular system may consistently cause a change in a PPG signal from patient to patient, the manner in which an individual patient's PPG signal changes may be influenced by the patient's unique peripheral vascular system characteristics and may provide a unique indication of that patient's disease state.

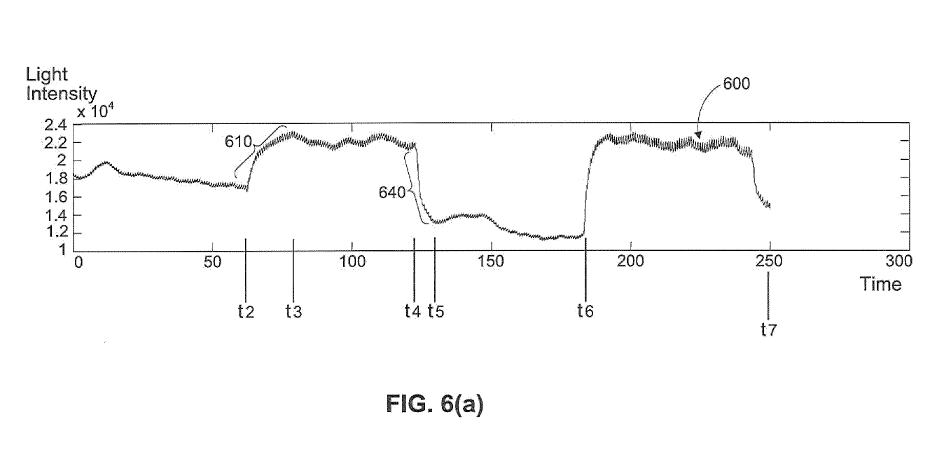

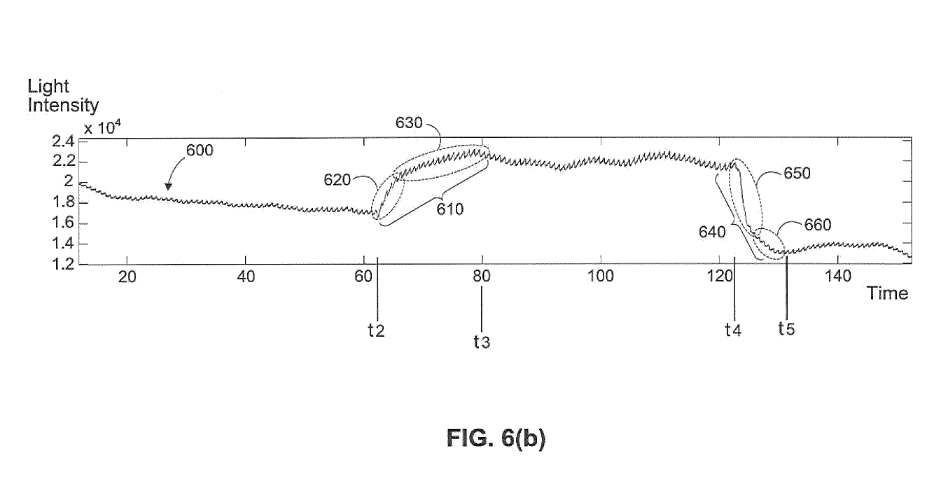

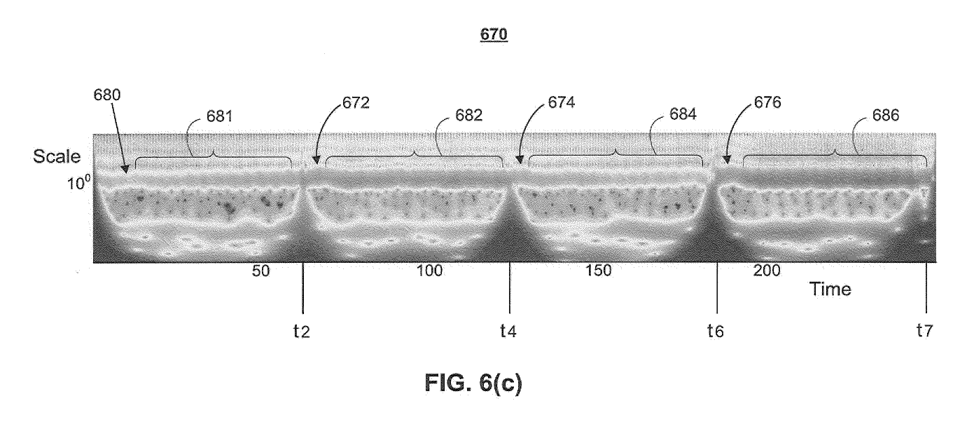

The extent to which PPG signal 500 changes, the period of time necessary for PPG signal 500 to change and then to return to either its initial baseline or a new baseline, and the shape of PPG signal 500 (including its individual pulses) during the transition period may provide information used to determine one or more characteristics of the patient's vascular system and to determine the patient's health status or disease state, FIG. 6(a) shows changes to PPG signal 600 induced by varying a load in the patient's vascular system in accordance with an embodiment. PPG signal 600 may increase from initial baseline and amplitude values at time t2, when the patient elevates the limb to which the sensor is coupled (e.g. sensor 12 coupled to the patient's finger) any suitable amount relative to his initial position (e.g., 0.5 meters above the patient's heart). In response to the limb elevation, the baseline of PPG signal 600 may increase in transition region 610 until time t3 (e.g., approximately 20 seconds later and the end of the transition period begun at time t2), after which time the baseline and amplitude of PPG signal 600 may each reach a new mean value.

At time t4, the patient may lower his limb relative to the elevated position and the initial position (e.g., the patient's arm and sensor 12 are lowered to a position 0.5 meters below the patient's heart) and the baseline of PPG signal 600 may decrease in transition region 640 until time t5, the end of the transition period, when the baseline and amplitude of PPG signal 600 may each reach a new mean value. The exercise may be repeated at time t6, as the patient elevates the same limb again (e.g., to a position 0.5 meters above the patient's heart), and at time t7, as the patient lowers the same limb to its initial resting position before time t2. As with the load exercises at times t2 and t4, the baseline and amplitude of PPG signal 600 do not change immediately at times t6 and t7 because one or more underlying static or dynamic characteristics of the patient's vascular system may influence the changes in PPG signal 600 during the transition regions.

FIG. 6(b) shows an enlarged portion of PPG signal 600 during its first two transition periods in accordance with an embodiment. Between times t2 and t3 and within transition region 610, the baseline of PPG signal 600 increases overall, but also increases at two different rates. The baseline increases rapidly in region 620, but increases more slowly in region 630 before PPG signal 600 peaks at t3 and the baseline settles, or oscillates, about a new mean baseline value. With an alteration of the load being induced (e.g., by lowering the limb) from the patient's vascular system at time t4 and during the ensuing transition region 640, the baseline of PPG signal 600 decreases rapidly in region 650, and decreases more slowly in region 660 before PPG signal 600 reaches a new local minimum at time t5 and the baseline oscillates about another mean baseline value.

The extent of and the rate of the baseline changes in regions 620, 630, 650, and 660, together with the transition periods t2 to t3 and t4 to t5, may be used to obtain one or more underlying static or dynamic characteristics of the patient's vascular system.

In some embodiments, microprocessor 48 or processor 412 may receive PPG signal 600 as an input along an input signal path and may obtain one or more characteristics that may be associated with, or may be mapped to, the observed changes. For example, the changes in PPG signal 600 may be used by system 10 or system 400 to determine the patient's vascular compliance, or the tendency of the patient's vessels to resist returning to their original dimensions when the induced load is removed from the patient's vascular system. The changes also may be used to determine the effectiveness with which the vascular system is able to drain venous blood from the patient's vessels. In some embodiments, the rapidity with which the patient's vessels constrict or dilate, or the patients vasotone, may be determined. For example, if the patient is younger and has a healthy vascular system, PPG signal 600 may rise relatively quickly in response to the limb being elevated and may overshoot its new baseline value and/or oscillate for a time before reaching its new baseline. If the patient is older or has a less healthy vascular system, a relatively slower, dampened increase in PPG signal 600 over a longer transition period may occur in response to elevating the limb. Further, the rate of change in the PPG during transition regions such as 610 and 640 may indicate venous drainage and/or compliance which may help diagnose venous hypertension. In addition, changes in pulse amplitude in PPG signal 600 may indicate changes in pulse pressure with respect to loading, which may in turn indicate cardiac output response to effort. Also, degree of notch washout in PPG signal 600 may indicate changes in peripheral resistance. In some embodiments, other changes in PPG signal 600 resulting from elevating the limb, such as the damping time needed for PPG signal 600 to stop oscillating around a new baseline value, the width of the pulses in PPG signal 600, the decay time or half-decay time of PPG signal 600, or the degree of the rise of the baseline, also may be used to obtain characteristics of the patient's vascular system and to provide further indication of the patient's current or future disease state or susceptibility.

One or more underlying characteristics of the patient's vascular system also may be determined by analyzing a wavelet transform of PPG signal 600. FIG. 6(c) shows an illustrative scalogram 670 derived from PPG signal 600 in accordance with an embodiment. In some embodiments, scalogram 670 may be derived by transforming PPG signal 600 using a continuous wavelet transform as described above with respect to equations (9) (14) and FIGS. 3(a)-3(b). Scalogram 670 may include pulse band 680 that is formed from the pulse component in PPG signal 600 producing a dominant band in wavelet space at a scale that corresponds to the pulse rate (e.g., approximately 60 beats per minute.) At points 672, 674 and 676, the patient's pulse rate may change and pulse band 680 may change in scale temporarily as a result of the patient physically exerting himself and inducing the load on his vascular system (e.g., elevating the limb at times t2 and t6) and also physically exerting himself and inducing a different load (e.g., lowering the limb at time t4). The energy contained in the pulse component of PPG signal 600, and by extension, pulse band 680, may change as a result of inducing the load, which causes increases or decreases in the amplitude of PPG signal 600. Between times t2 and t4 (region 682), and between times t6 and t7 (region 686), when the limb is elevated relative to its initial rest position in region 681, the energy in pulse band 680 increases, as shown by the deepened coloration in pulse band 680. Between times t4 and t6 (region 684), when the limb is lowered relative to its initial rest position in region 681, the energy in pulse band 680 decreases, as shown by the lessened coloration in pulse band 680. This is consistent with the underlying changes in PPG signal 600, in which the baseline means increases and decreases, respectively.

In some embodiments, scalogram 670 may be derived by microprocessor 48 or processor 412 and may be further analyzed to obtain underlying vascular characteristics from the increased heart rate and amplitude changes observed in scalogram 670.

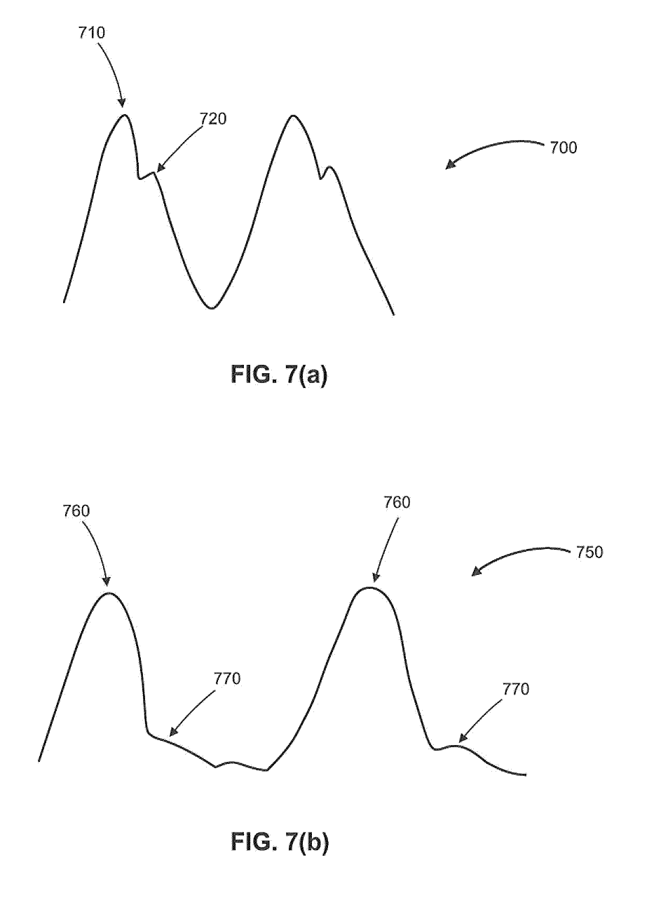

In some embodiments, one or more underlying characteristics of the patient's vascular system also may influence the morphology of individual PPG signal pulses in response to an induced load on the vascular system. FIGS. 7(a)-(b) show graphical illustrations of a change in PPG signal 700 induced by a load in the patient's vascular system in accordance with an embodiment. In FIG. 7(a), PPG signal 700 may include primary peak 710 and dicrotic notch 720 when the patient is at rest (e.g., the patient's arm is at the level of his heart). When the patient elevates that limb above its rest position in FIG. 7(b), the morphology of each pulse in PPG signal 750 may change as dicrotic notch 770 may wash out and the initial pulse component of each pulse, including primary peak 760, may dominate over the reflected portion of each pulse. The new morphology of PPG signal 760 when the limb is elevated may be used by microprocessor 48 or processor 412 to determine the morphology characteristics of the patient's vascular system and to determine the patient's current or future health status or disease state. For example, the degree of dicrotic notch 770 washout may be analyzed to obtain the patient's venous compliance.

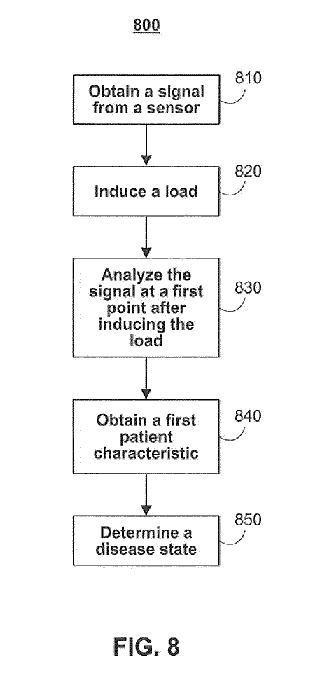

FIG. 8 is a flow chart of an illustrative process for determining a disease state using an induced load in accordance with an embodiment. Process 800 may begin at step 810, when a patient signal (e.g., PPG signal 600) may be obtained from any suitable sensor, such as sensor 12, coupled to a patient at rest. Process 800 may advance to step 820, where a load may be induced on a patient that may cause a change in the sensed signal. For example, patient 40 may elevate his limb to which sensor 12 may be coupled to a position above the initial resting position of the limb. The elevation may induce a load on the vascular system of patient 40, which may change the PPG signal being sensed. In some embodiments, the patient may induce a different load on his vascular system by lowering the limb relative to its initial resting position.

In some embodiments, process 800 may advance to step 830, where the sensed signal may be analyzed at at least one point following the introduction of the load. For example, microprocessor 48 or processor 412 may analyze multiple points of PPG signal 600 following the induction of the load on the patients vascular system for changes such as the rate of change of PPG signal 600, the overall change of the baseline and/or amplitude of PPG signal 600, or the morphology of the change in PPG signal 600 and/or its individual pulses. At step 840, at least a first patient characteristic may be obtained or determined as a result of the analysis of the changed signal at step 830. For example, microprocessor 48 or processor 412 may map a poor rate of change of PPG signal 600 analyzed at step 830 to a particular vascular system characteristic, such as poor patient vasotone, using a process or a look-up table stored in microprocessor 48 or processor 412. In some embodiments, the patient characteristic may be displayed or otherwise communicated to a user of system 10/400 or the patient. For example, the patient characteristic may be displayed on display 28 or output 414. Alternatively or in addition, the patient characteristic may be communicated in the form of an audible tone or message generated by system 10/400 that may be heard by the user or may be communicated to a user at a remote location (e.g., at a nurse's station outside of the patient's room).

In some embodiments, process 800 may advance to step 850, where the patient characteristic obtained in step 840 may be used to determine at least one disease state of the patient. For example, a patient characteristic of poor patient vasotone, as determined by microprocessor 48 or processor 412 at step 840, may be associated with a disease state of septicemia, and the associated disease state may be displayed or otherwise communicated to a user of system 10/400 or the patient in any suitable manner. For example, as with the patient characteristic, the disease state may be displayed on display 28 or output 414, and/or may be communicated in the form of an audible tone or message generated by system 10/400.

In some embodiments, the patient characteristic obtained at step 840 and/or the disease state determined at step 850 may be used to generate and store an alarm in system 10/400. For example, the patient characteristic and/or the disease state may indicate that the patient is experiencing a medical emergency that requires immediate treatment. The alarm may be generated by microprocessor 48 or processor 412 to alert a user of system 10/400 in any suitable fashion, including a visual cue or message on display 28 or output 414, an audible cue or tone, or a message that may be communicated on any suitable output device coupled to system 10/400 (e.g., a display at a nurse's station). In some embodiments, the alarm may be analyzed by microprocessor 48 or processor 412 to provide historical information about the patient's health or trends in the patient's vascular system. For example, when an alarm is generated in response to obtaining a particular patient characteristic or determining a particular disease state, a marker or indicator also may be generated by microprocessor 48 or processor 412 and stored (e.g., stored in RAM 54 or ROM 52) as being associated with the patient information (e.g., PPG signal 600) obtained at a certain point in time. Each alarm may be analyzed to generate and store a new marker, and the markers may be further analyzed to indicate or study trends in the patient's vascular system and/or disease state. The marker may be displayed on display 28 or output 414 at the time that the alarm is used to alert the user of system 10/400 or it may be stored in RAM 54 or ROM 52 for future recall by the user.

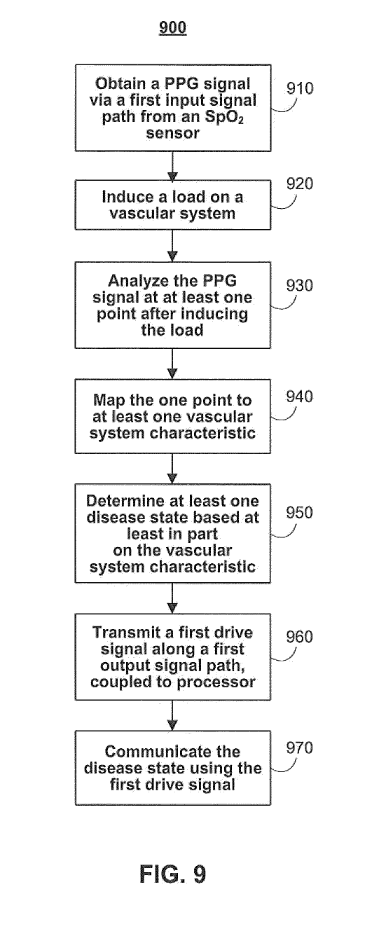

FIG. 9 is a flow chart of an illustrative process for determining a disease state in a vascular system using a PPG signal in accordance with an embodiment. Process 900 may begin at step 910, where a PPG signal may be obtained along a first input signal path (e.g., cable 24) by any suitable system, such as system 10, using a pulse oximetry sensor (e.g., sensor 12) coupled to a patient. In some embodiments, process 900 may advance to step 920, where any suitable load, such as a limb elevation or limb lowering, may be induced on the patient's vascular system. Inducing the load in the vascular system may cause at least one change in the PPG signal obtained by system 10.

In some embodiments, process 900 may advance to step 930, where the PPG signal may be analyzed at at least one point after the load was induced (e.g., during transition region 610). For example, microprocessor 48 or processor 412 may analyze multiple points following the induction of the load, to analyze the PPG signal's rate of change, the overall change of the baseline and/or amplitude of the PPG signal, or the morphology of the change in the individual PPG signal pulses. In some embodiments, microprocessor 48 or processor 412 also may compare the analyzed points against previous PPG signal points that were obtained before the load was induced and were stored in RAM 54 or ROM 52 of system 10. This comparison may be further analyzed, for example, to determine the overall change of the PPG signal.

In some embodiments, process 900 may advance to step 940, in which the one or more points analyzed by microprocessor 48 or processor 412 at step 930 may be mapped to, or associated with, at least one vascular system characteristic of the patient. For example, microprocessor 48 or processor 412 may map the points analyzed after the load was induced on the vascular system to one or more vascular system characteristics using a process stored in microprocessor 48 or processor 412 into which process the analyzed points may be inserted, or using a look-up table containing information about characteristics associated with the analyzed points. In some embodiments, microprocessor 48 or processor 412 also may use one or more PPG signal points obtained before the load was induced and stored in system 10 to further associate at least one vascular system characteristic with the PPG signal points obtained before and after the load was induced. In some embodiments, the vascular system characteristic(s) that may be associated with the analyzed points at step 940 may be displayed or otherwise communicated to a user of system 10 or the patient in any suitable manner.

In some embodiments, process 900 may advance to step 950, in which at least one disease state may be determined using the vascular system characteristic(s) obtained or mapped to in step 940. For example, the vascular system characteristic(s) that were obtained as a result of analyzing the PPG signal points that occurred after (and in some instances, before) the load was induced may also indicate a current or future health status or disease state of the patient. Microprocessor 48 or processor 412 may determine a disease state at least in part by the obtained vascular system characteristic(s). In some embodiments, process 900 may advance to step 960, where a first drive signal containing information related to the determined disease state is transmitted by microprocessor 48 or processor 412 along a first output signal path, and step 970, where the disease state may be communicated (e.g. on display 28) using the first drive signal.

It will be understood that the foregoing is only illustrative of the principles of the disclosure, and that the disclosure can be practiced by other than the described embodiments, which are presented for purposes of illustration and not of limitation.

What is claimed is:

1. A method for communicating a disease state of a patient, the method comprising:
obtaining a signal from a patient sensor, wherein the signal changes in response to inducing a load on a vascular system of the patient;
analyzing the signal at a first point to obtain a first vascular system characteristic, wherein the first point occurs after the load is induced on the vascular system and wherein the signal is analyzed by a processor coupled to the patient sensor;
determining the disease state based at least in part on the first vascular system characteristic; and
communicating the disease state to an output device.

2. The method of claim 1, wherein the inducing the load comprises changing an elevation of a limb to which the patient sensor is coupled.

3. The method of claim 1, wherein the first vascular system characteristic is obtained based at least in part on a time period between the inducing the load and the first point occurring after the load is induced.

4. The method of claim 1, wherein the first point occurs while the signal is changing in response to inducing the load.

5. The method of claim 1, wherein the signal is a photoplethysmograph signal.

6. The method of claim 5, wherein the first point occurs within a dicrotic notch of a pulse of the photoplethysmograph signal.

7. The method of claim 5, further comprising:
transforming using the processor the photoplethysmograph signal into a transformed signal using a continuous wavelet transform;
generating using the processor a scalogram based at least in part on the transformed signal;
identifying using the processor a band on the scalogram; and
obtaining the first vascular system characteristic based at least in part on the band.

8. The method of claim 1, wherein the first vascular system characteristic is one of the group consisting of vasotone, venous compliance, and/or the ability of venous blood to drain, and/or combinations thereof.

9. The method of claim 1, further comprising:
analyzing the signal at the first point to obtain a second vascular system characteristic; and
determining the disease state based at least in part on the first and second vascular system characteristics.

10. The method of claim 1, further comprising:
generating an alarm based at least in part on the disease state; and
communicating the alarm to the output device.

11. A system for communicating a disease state of a patient, the system comprising:
an input signal generator for generating a signal, wherein the signal changes based at least in part upon inducing a load on a vascular system of the patient;
a processor coupled to the input signal generator, wherein the processor is capable of:
analyzing the signal at a first point to obtain a first vascular system characteristic, wherein the first point occurs after the load is induced on the vascular system;
determining the disease state based at least in part on the first vascular system characteristic; and
an output device coupled to the processor, wherein the output device is capable of communicating the disease state.

12. The system of claim 11, wherein the inducing the load comprises changing an elevation of a limb to which the input signal generator is coupled.

13. The system of claim 11, wherein the processor is further capable of obtaining the first vascular system characteristic based at least in part on a time period between the inducing the load and the first point occurring after the load is induced.

14. The system of claim 11, wherein the first point occurs while the signal is changing in response to inducing the load.

15. The system of claim 11, wherein the signal is a photoplethysmograph signal.

16. The system of claim 15, wherein the first point occurs within a dicrotic notch of a pulse of the photoplethysmograph signal.

17. The system of claim 15, wherein the processor is further capable of:
transforming the photoplethysmograph signal into a transformed signal using a continuous wavelet transform;
generating a scalogram based at least in part on the transformed signal;
identifying a band on the scalogram; and
obtaining the first vascular system characteristic based at least in part on the band.

18. The system of claim 11, wherein the first vascular system characteristic is one of the group consisting of vasotone, venous compliance, and/or the ability of venous blood to drain, and/or combinations thereof.

19. The system of claim 11, wherein the processor is further capable of:
analyzing the signal at the first point to obtain a second vascular system characteristic; and
determining the disease state based at least in part on the first and second vascular system characteristics.

20. A computer-readable medium capable of communicating a disease state of a patient, the computer-readable medium having computer program instructions recorded thereon, which if activates would cause a processor to:
obtain a signal from a patient sensor, wherein the signal changes in response to inducing a load on a vascular system of the patient;
analyze the signal at a first point to obtain a first vascular system characteristic, wherein the at least a first point occurs after the load is induced on the vascular system;
determine the disease state based at least in part on the first vascular system characteristic; and
communicating the disease state to an output device.
